(19) United States
(12) Patent Application Publication
(10) Pub. No.: US 2026/0347331 A1
(43) Pub. Date: Nov. 5, 2026

(54) PREPARATION OF OPTICALLY ACTIVE ENANTIOMERS OF PIPERONYL AMINES BY RESOLUTION OF THE DIASTEREOMERIC SALTS

(71) Applicant: EmpathBio, Inc., New York, NY (US)

(72) Inventors: Luigi ANZALONE, Hull, MA (US); Nicholas MORRA, Ontario (CA); Majed FAWAZ, Foxborough, MA (US)

(21) Appl. No.: 19/727,853

(22) Filed: Jul. 1, 2026

Related U.S. Application Data
(63) Continuation of application No. 17/946,406, filed on Sep. 16, 2022, now Pat. No. 12,715,852.
(60) Provisional application No. 63/245,496, filed on Sep. 17, 2021.

(51) Int. Cl. C07D 317/06

(52) U.S. Cl. CPC C07D 317/06

(57) ABSTRACT

Provided herein is a process for resolving the (S)- and (R)-enantiomers of a compound of Formula (I), wherein L, R¹, and R² are defined herein.

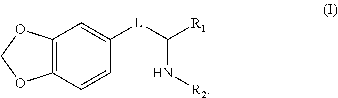

### CROSS-REFERENCE TO RELATED APPLICATION

The present application is a Continuation of U.S. application Ser. No. 17/946,406, filed Sep. 16, 2022, which claims the benefit of priority to U.S. Provisional Application No. 63/245,496, filed Sep. 17, 2021, the contents of which are hereby incorporated by reference in its entirety.

### BACKGROUND

3,4-methylenedioxymethamphetamine (MDMA) is considered the prototype of a class of compounds called entactogens, which means “to touch within”, their main characteristic being their ability to increase feelings of love, empathy and closeness towards others. Structurally, MDMA is a ring-substituted phenethylamine with a chiral molecular center that gives rise to two stereoisomers: S-(+)-MDMA and R-(−)-MDMA. Typically, effects of the former resemble those of psychostimulants and are primarily mediated by dopaminergic and noradrenergic pathways, including increases in motor activity and euphoria, whereas the latter induces qualitative effects similar to classical psychedelics, such as ego-dissolution and perceptive alterations, mediated by serotonergic pathways, including direct 5-HT2A receptor agonism. The molecular mechanisms for these differences are supported by preclinical evidence and point to a higher therapeutic index for the R-enantiomer.

There remains a need for improved processes of making enantiomers of 3,4-methylenedioxymethamphetamine and analogs thereof.

### SUMMARY

In an aspect, the present disclosure provides a process for resolving enantiomers of piperonyl amines.

In some embodiments, the present disclosure provides a process for resolving the (R) or (S) enantiomers of 3,4-methylenedioxymethamphetamine (MDMA).

In embodiments, provided herein is a process for resolving the (S)- and (R)-enantiomers of a compound of Formula (I):

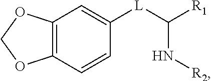

wherein L is a C₁-C₅ alkylene, carbocyclyl, or heterocyclyl;
R¹ is selected from the group consisting of alkyl, benzyl and phenethyl; and
R² is selected from the group consisting of hydrogen, alkyl, benzyl and phenethyl;
the process comprising:
(a) mixing a compound of Formula (I) and a chiral acid in a solvent to provide a diastereomeric salt of Compound (I) and
(b) resolving the (S)- and (R)-enantiomers of the compound of Formula (I),
wherein the enantiomeric excess of the compound of Formula (I) is less than about 50%.

In embodiments, the resolution (b) comprises preferential crystallization of the (S)- or (R)-enantiomer of the compound of Formula (I).

In embodiments, the chiral acid is selected from the group consisting of (S)-2-methoxy-2-phenylacetic acid, L-tartaric acid, dibenzoyl-L-tartaric acid, di-p-toluoyl-L-tartaric acid, L-ascorbic acid, L-aspartic acid, (S)-(+)-mandelic acid, (−)-di-O-isopropyledene-2-keto-L-gulonic acid and (1R, 3S)-(+)-camphoric acid,

In embodiments, the chiral acid is selected from the group consisting of (R)-2-methoxy-2-phenylacetic acid, D-tartaric acid, dibenzoyl-D-tartaric acid, di-p-toluoyl-D-tartaric acid, D-ascorbic acid, D-aspartic acid, (R)-(−)-mandelic acid, and (1S, 3R)-(−)-camphoric acid.

In embodiments, the chiral acid is (S)-2-methoxy-2-phenylacetic acid.

### DETAILED DESCRIPTION

Throughout this disclosure, various patents, patent applications and publications are referenced. The disclosures of these patents, patent applications and publications in their entireties are incorporated into this disclosure by reference for all purposes to describe the state of the art more fully as known to those skilled therein as of the date of this disclosure. This disclosure will govern in the instance that there is any inconsistency between the patents, patent applications and publications cited and this disclosure.

### DEFINITIONS

For convenience, certain terms employed in the specification, examples and claims are collected here. Unless defined otherwise, all technical and scientific terms used in this disclosure have the same meanings as commonly understood by a person skilled in the art to which this disclosure belongs.

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example, in a list of numerical values such as “about 49, about 50, about 55, . . . ”, “about 50” means a range extending to less than half the interval(s) between the preceding and subsequent values, e.g., more than 49.5 to less than 50.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

The term “pharmaceutically acceptable salts” includes both acid addition salts. Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, amino acids (such as glycine, alanine, arginine, -asparagine, aspartic acid, cysteine, glutamine, glutamic acid, histidine, isoleucine, leucine, lysine, methionine, phenylalanine, proline, serine, threonine, tryptophan, tyrosine, value), etc. The acids that may be used to prepare pharmaceutically acceptable acid addition salts of such basic compounds are those that form non-toxic acid addition salts, i.e., salts containing pharmaceutically acceptable anions, including but not limited to malate, oxalate, chloride, bromide, iodide, nitrate, acetate, tartrate, oleate, fumarate, formate, benzoate, glutamate, methanesulfonate, benzenesulfonate, and p-toluenesulfonate salts. Those skilled in the art will further recognize that acid addition salts may be prepared by reaction of the compounds with the appropriate inorganic or organic acid via any of a number of known methods.

When a range of values is listed, it is intended to encompass each value and sub-range within the range. For example, “C1-C6 alkyl” is intended to encompass C1, C2, C3, C4, C5, C6; C1-6, C1-5, C1-4, C1-3, C1-2, C2-6, C2-5, C2-4, C2-3, C3-6, C3-5, C3-4, C4-6, C4-5, and C5-6 alkyl.

“Alkyl” or “alkyl group” refers to a fully saturated, straight or branched hydrocarbon chain having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight, or branched divalent hydrocarbon chain radical, and having from one to twelve carbon atoms. Non-limiting examples of C₁-C₁₂ alkylene include methylene, ethylene, propylene, n-butylene, and the like. The alkylene chain is attached to the rest of the molecule through a single bond and to a radical group (e.g., those described herein) through a single bond. The points of attachment of the alkylene chain to the rest of the molecule and to the radical group can be through one carbon or any two carbons within the chain. Unless stated otherwise specifically in the specification, an alkylene chain can be optionally substituted.

“Carbocyclyl,” “carbocyclic ring” or “carbocycle” refers to a rings structure, wherein the atoms which form the ring are each carbon, and which is attached to the rest of the molecule by a single bond. Carbocyclic rings can comprise from 3 to 20 carbon atoms in the ring. Carbocyclic rings include e.g., aryls and cycloalkyl as defined herein. Unless stated otherwise specifically in the specification, a carbocyclyl group can be optionally substituted.

“Aryl” refers to a hydrocarbon ring system comprising hydrogen, 6 to 18 carbon atoms and at least one aromatic ring, and which is attached to the rest of the molecule by a single bond. For purposes of this disclosure, the aryl can be a monocyclic, bicyclic, tricyclic or tetracyclic ring system, which can include fused or bridged ring systems. Aryls include, but are not limited to, aryls derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the “aryl” can be optionally substituted.

“Cycloalkyl” refers to a stable non-aromatic monocyclic or polycyclic fully saturated hydrocarbon consisting solely of carbon and hydrogen atoms, which can include fused, bridged, or spirocyclic ring systems, having from three to twenty carbon atoms (e.g., having from three to ten carbon atoms) and which is attached to the rest of the molecule by a single bond. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Heterocyclyl,” “heterocyclic ring” or “heterocycle” refers to a stable saturated, unsaturated, or aromatic 3- to 20-membered ring which consists of two to nineteen carbon atoms and from one to six heteroatoms selected from the group consisting of nitrogen, oxygen and sulfur, and which is attached to the rest of the molecule by a single bond. Heterocyclyl or heterocyclic rings include heteroaryls, heterocyclylalkyls, heterocyclylalkenyls, and hetercyclylalkynyls. Unless stated otherwise specifically in the specification, the heterocyclyl can be a monocyclic, bicyclic, tricyclic or tetracyclic ring system, which can include fused, bridged, or spirocyclic ring systems; and the nitrogen, carbon or sulfur atoms in the heterocyclyl can be optionally oxidized; the nitrogen atom can be optionally quaternized; and the heterocyclyl can be partially or fully saturated. Examples of such heterocyclyl include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted.

The term “substituted” used herein means any of the groups described herein (e.g., alkyl, heterocyclyl, and/or heteroaryl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

### Methods

While the racemate is the most commonly administered form of MDMA currently, recent research suggests that there are distinct differences in the pharmacology of the two enantiomers. Hence, the development of efficient asymmetric strategies for producing enantiopure MDMA and other piperonyl amines is of high importance.

Enantiomers of MDMA have been separated via the use of removable chiral auxiliaries, however the use of chiral auxiliaries necessitates multiple chemical steps to install, utilize, and cleave the auxiliary which is required in stoichiometric quantities resulting in poor step- and atom-economy. Lourenco et al, J Pharmaceutical and Biomedical Analysis, 2013, 73, 13-17 were able to separate enantiomers of MDMA by batch chromatography with peak shaving and recycling. However, there are multiple disadvantages associated with chromatography, such as requirement for repetitive extractions, large volumes of organic-based mobile phase for component elution, and the generation of significant solvent waste and disposal costs.

This present disclosure describes methods for the preparation of optically active enantiomers of compounds of Formula (I):

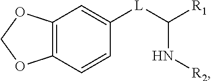

wherein L, R¹, and R² are defined herein, by resolution of its diastereomeric salts.

Using the methods disclosed herein enantiomers of compounds of Formula (I), such as MDMA can be obtained in high enantiomeric purity. The present methods offer advantages over prior routes, where the resolution of racemic MDMA via selective crystallization of diastereomeric salt proved to be an ineffective method for synthesizing MDMA in high enantiomeric excess (see e.g., Dunlap et al, ACS Chem Neuroscience, 2018, 9(10), 2408-2427, and Pizarro et al, Bioorganic and Medicinal Chemistry, 2002, 10, 1085-1092). The methods disclosed herein are also superior to both the chromatography and the chiral auxiliary methods for the ease of operation, the cost effectiveness, and the ability to recycle the chiral resolving acid, and utilize racemic MDMA which is readily available.

In embodiments, optically active enantiomers of compounds disclosed herein, e.g., compounds of Formula (I) display an improved pharmacological profile compared to the corresponding racemic mixtures. In embodiments, the resolution methods described herein are performed by combining a racemic mixture of enantiomers of the amine and a resolving acid with a defined stereospecificity in a solvent to form a solution and said resolving acid is capable of binding with at least one of the enantiomers to form a crystalline precipitate. This single diastereomeric solid can then be freebased by treating with a base to provide the optically active enantiomer. In embodiments, if the other enantiomer is desired, then the filtrate can be concentrated to a residue and the resulting opposite diastereomer is freebased as previously to provide the desired compound.

The present disclosure provides a process for resolving the (S)- and (R)-enantiomers of a compound of Formula (I):

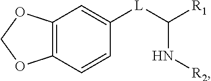

wherein L is a C₁-C₅ alkylene, carbocyclyl, or heterocyclyl;
R¹ is selected from the group consisting of alkyl, benzyl and phenethyl; and
R² is selected from the group consisting of hydrogen, alkyl, benzyl and phenethyl;
the process comprising:
(a) mixing a compound of Formula (I) and a chiral acid in a solvent to provide a diastereomeric salt of Compound (I) and
(b) resolving the (S)- and (R)-enantiomers of the compound of Formula (I),
wherein the enantiomeric excess of the compound of Formula (I) is less than about 50%.

In some embodiments of the compounds of Formula (I), R¹ is selected from the group consisting of alkyl, benzyl and phenethyl.

In some embodiments of the compounds of Formula (I), R¹ is alkyl.

In some embodiments of the compounds of Formula (I), R¹ is benzyl.

In some embodiments of the compounds of Formula (I), R¹ is phenethyl.

In some embodiments of the compounds of Formula (I), R² is selected from the group consisting of hydrogen, alkyl, benzyl and phenethyl.

In some embodiments of the compounds of Formula (I), R² is hydrogen.

In some embodiments of the compounds of Formula (I), R² is alkyl.

In some embodiments of the compounds of Formula (I), R² is benzyl.

In some embodiments of the compounds of Formula (I), R² is phenethyl.

In some embodiments of the compounds of Formula (I), R¹ and R² are independently C₁₋₆ alkyl.

In some embodiments of the compounds of Formula (I), R¹ and R² are independently C₁₋₃ alkyl.

In some embodiments of the compounds of Formula (I), R¹ and R² are methyl.

In some embodiments of the compounds of Formula (I), R¹ is C₁₋₆ alkyl and R² is hydrogen.

In some embodiments of the compounds of Formula (I), R¹ is C₁₋₃ alkyl and R² is hydrogen.

In some embodiments of the compounds of Formula (I), R¹ is methyl and R² is hydrogen.

In some embodiments of the compounds of Formula (I), L is methylene.

In some embodiments of the compounds of Formula (I), L is C₁₋₆ alkylene.

In some embodiments of the compounds of Formula (I), L is C₁₋₃ alkylene.

In some embodiments of the compounds of Formula (I), L is —(CH₂)ₖ—, wherein k is 1, 2, or 3.

In some embodiments of the compounds of Formula (I), L is —CH₂—.

In some embodiments of the compounds of Formula (I), R¹ and R² are methyl and L is methylene.

In some embodiments of the compounds of Formula (I), R¹ is methyl and R² is hydrogen and L is methylene.

In some embodiments, the solvent is selected from the group consisting of toluene, acetonitrile, methanol, ethanol, isopropanol, dichloromethane, and mixtures thereof.

In some embodiments, the solvent is an aromatic solvent e.g., toluene.

In some embodiments, the solvent is an alcohol e.g., methanol, ethanol or isopropanol.

In some embodiments, the solvent is a chlorinated solvent e.g., dichloromethane, dichloroethane and the like.

In some embodiments, the solvent is an ether solvent such as, tetrahydrofuran, dioxane, diethyl ether.

In some embodiments, the solvent is ethyl acetate.

In some embodiments, the solvent is acetonitrile.

In some embodiments, step (a) comprises mixing a compound of Formula (I) and a chiral acid in a solvent while maintaining the temperature at about 20 to 25 °C.

In some embodiments, the solution of a compound of Formula (I) and the chiral acid is mixed for about 0.5-48 hours, including about 0.5 hours, about 1 hours, about 2 hours, about 3 hours, about 4 hours, about 5 hours, about 6 hours, about 7 hours, about 8 hours, about 9 hours, about 10 hours, about 12 hours, about 14 hours, about 16 hours, about 18 hours, about 20 hours, about 22 hours, about 24 hours, about 26 hours, about 28 hours, about 30 hours, about 32 hours, about 34 hours, about 36 hours, about 38 hours, about 40 hours, about 42 hours, about 44 hours, about 46 hours, to about 48 hours or more, including all subranges and values therebetween.

In some embodiments, the mixture of step (a) further comprises an anti-solvent. In some embodiments, the anti-solvent is a hydrocarbon solvent or an ether solvent.

In some embodiments, the anti-solvent is selected from the group consisting of heptanes and methyl t-butyl ether.

In some embodiments, the anti-solvent is hexane, pentane, or heptane.

In some embodiments, the Step (a) mixture comprises about 0.5 molar equivalents of the chiral acid relative to amount of racemate.

In some embodiments the Step (a) mixture comprises about 1.0 molar equivalents of the chiral acid relative to amount of racemate.

In some embodiments, the chiral acid is selected from the group consisting of(S)-2-methoxy-2-phenylacetic acid, L-tartaric acid, dibenzoyl-L-tartaric acid, di-p-toluoyl-L-tartaric acid, L-ascorbic acid, L-aspartic acid, (S)-(+)-mandelic acid, (−)-di-O-isopropyledene-2-keto-L-gulonic acid and (1R, 3S)-(+)-camphoric acid. In some embodiments, the chiral acid is (S)-2-methoxy-2-phenylacetic acid.

In some embodiments, the chiral acid is (S)-2-methoxy-2-phenylacetic acid, L-tartaric acid, or dibenzoyl-L-tartaric acid.

In some embodiments, the chiral acid is (S)-2-methoxy-2-phenylacetic acid. In embodiments, the chiral acid is L-tartaric acid.

In embodiments, the chiral acid is dibenzoyl-L-tartaric acid.

In embodiments, the chiral acid is di-p-toluoyl-L-tartaric acid.

In embodiments, the chiral acid is L-ascorbic acid.

In embodiments, the chiral acid is L-aspartic acid.

In embodiments, the chiral acid is (S)-(+)-mandelic acid.

In embodiments, the chiral acid is (−)-di-O-isopropyledene-2-keto-L-gulonic acid.

In embodiments, the chiral acid is and (1R, 3S)-(+)-camphoric acid.

In some embodiments, the chiral acid is selected from the group consisting of (R)-2-methoxy-2-phenylacetic acid, D-tartaric acid, dibenzoyl-D-tartaric acid, di-p-toluoyl-D-tartaric acid, D-ascorbic acid, D-aspartic acid, (R)-(−)-mandelic acid, (+)-di-O-isopropyledene-2-keto-D-gulonic acid and (1S, 3R)-(−)-camphoric acid. In some embodiments, the chiral acid is (R)-2-methoxy-2-phenylacetic acid.

In some embodiments, the chiral acid is (R)-2-methoxy-2-phenylacetic acid.

In embodiments, the chiral acid is D-tartaric acid.

In embodiments, the chiral acid is dibenzoyl-D-tartaric acid.

In embodiments, the chiral acid is di-p-toluoyl-D-tartaric acid.

In embodiments, the chiral acid is D-ascorbic acid.

In embodiments, the chiral acid is D-aspartic acid.

In embodiments, the chiral acid is (R)-(−)-mandelic acid.

In embodiments, the chiral acid is (+)-di-O-isopropyledene-2-keto-D-gulonic acid.

In embodiments, the chiral acid is and (1S, 3R)-(−)-camphoric acid.

In some embodiments, the chiral acid is (R)-2-methoxy-2-phenylacetic acid, D-tartaric acid, or dibenzoyl-D-tartaric acid.

In some embodiments of the methods disclosed herein, the diastereomeric chiral salt precipitate comprises the (R)-enantiomer of the compound of Formula (I).

In some embodiments of the methods disclosed herein, the diastereomeric chiral salt precipitate comprises the (S)-enantiomer of the compound of Formula (I).

In some embodiments of the methods of resolving a compound of Formula (I), the method further comprises isolating the preferentially crystallized diastereomeric salt of the compound of Formula (I). In some embodiments, the diastereomeric salt is isolated by filtration.

In some embodiments, after resolving the compound of Formula (I), the other enantiomer (i.e. the enantiomer which does not form a chiral salt with the resolving agent) can be recovered from the mother liquor derived from the resolution step.

In some embodiments of the methods disclosed herein, mixing a compound of Formula (I) and a chiral acid in a solvent provides diastereomeric salt precipitate of Formula (I-a):

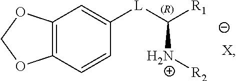

wherein X is the conjugate base of a chiral acid, e.g., as described herein. In some embodiments, the diastereomeric salt precipitate of Formula (I-a) is isolated by filtration and the mother liquor concentrated to give a compound of Formula (1-b).

In some embodiments of the methods disclosed herein, mixing a compound of Formula (I) and a chiral acid in a solvent provides diastereomeric salt precipitate of formula:

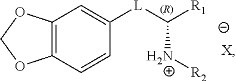

wherein X is the conjugate base of a chiral acid, e.g., as described herein. In some embodiments, the diastereomeric salt precipitate of Formula (I-b) is isolated by filtration and the mother liquor concentrated to give a compound of Formula (1-a).

In some embodiments of the methods disclosed herein, mixing a compound of Formula (I) and a chiral acid in a solvent provides diastereomeric salt precipitate of formula:

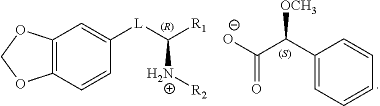

In some embodiments of the methods disclosed herein, mixing a compound of Formula (I) and a chiral acid in a solvent provides diastereomeric salt precipitate of formula:

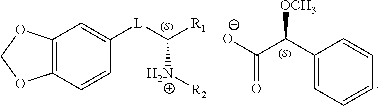

In some embodiments of the methods of resolving a compound of Formula (I), the method further comprises combining the crystallized diastereomeric salt and a base to provide enantioenriched free base of the (S)- or (R)-enantiomer of the compound of Formula (I).

In some embodiments, the base is selected from the group consisting of hydroxides, alkoxides (e.g., methoxide, ethoxide), alkali metal amides (e.g., lithium diisopropylamide), hydrides (e.g., NaH), organolithium, or Grignard reagents. For bases that require a counterion, exemplary counterions include alkali metals, alkaline earth metals, e.g., Li⁺, Na⁺, K⁺, Ca²⁺, Mg²⁺, or organic counterions like a tetra-alkyl ammonium. In some embodiments, the base is a hydroxide base. In some embodiments, the base is sodium hydroxide.

In some embodiments, the resolution (b) comprises preferential crystallization of the (S)- or (R)-enantiomer of the compound of Formula (I).

In some embodiments, the diastereomeric salt comprising the (S)-enantiomer of the compound of Formula (I) is preferentially crystallized.

In some embodiments, the diastereomeric salt comprising the (R)-enantiomer of the compound of Formula (I) is preferentially crystallized.

In some embodiments, the compound of Formula (I) has an enantiomeric excess (“ee”) of less than about 50% ee before the compound of Formula (I) is resolved by the methods disclosed herein, less than about 45% before the compound of Formula (I) is resolved, less than about 40% before the compound of Formula (I) is resolved, less than about 35% before the compound of Formula (I) is resolved, less than about 30% before the compound of Formula (I) is resolved, less than about 30% before the compound of Formula (I) is resolved, less than about 25% before the compound of Formula (I) is resolved, less than about 20% before the compound of Formula (I) is resolved, less than about 15% before the compound of Formula (I) is resolved, less than about 10% before the compound of Formula (I) is resolved, less than about 5% before the compound of Formula (I) is resolved, including all subranges and values therebetween. In some embodiments the compound of Formula (I) is a racemic compound before the compound of Formula (I) is resolved by the methods disclosed herein. In some embodiments, ee is measured by chiral HPLC.

In some embodiments, the methods of resolving a the compound of Formula (I) described herein, provides the (S)-enantiomer of a compound of Formula (I) an enantiomeric excess of about or at least about 60% ee, about or at least about 65% ee, about or at least about 70% ee, about or at least about 80% ee, about or at least about 85% ee, about or at least about 90% ee, about or at least about 91%, about or at least about 92%, about or at least about 93% ee, about or at least about 94% ee, about or at least about 95% ee, about or at least about 96% ee, about or at least about 97% ee, about or at least about 98% ee, about or at least about 99% ee, about or at least about 99.5% ee, or about or at least about 99.9% ee, including all values therebetween. In some embodiments, the process provides the (S)-enantiomer of a compound of Formula (I) in an enantiomeric excess of about or at least about 80% ee. In some embodiments, the process provides the (S)-enantiomer of a compound of Formula (I) in an enantiomeric excess of a least about 99.5%. In some embodiments, ee is measured by chiral HPLC.

In some embodiments, the methods of resolving a the compound of Formula (I) described herein, provides the (S)-enantiomer of a compound of Formula (I) an enantiomeric excess of from about 60% ee to about 100% ee, including from about 65% ee, about 70% ee, about 80% ee, about 85% ee, about 90% ee, about 91%, about 92%, about 93% ee, about 94% ee, about 95% ee, about 96% ee, about 97% ee, about 98% ee, about 99% ee, about 99.5% ee, about 99.9% ee to about 100% ee, including all subranges therebetween.

In some embodiments, the methods of resolving a the compound of Formula (I) described herein, provides the (R)-enantiomer of a compound of Formula (I) an enantiomeric excess of about or at least about 60% ee, about or at least about 65% ee, about or at least about 70% ee, about or at least about 80% ee, about or at least about 85% ee, about or at least about 85% ee, about or at least about 90% ee, about or at least about 91%, about or at least about 92%, about or at least about 93% ee, about or at least about 94% ee, about or at least about 95% ee, about or at least about 96% ee, about or at least about 97% ee, about or at least about 98% ee, about or at least about 99% ee, about or at least about 99.5% ee, or about or at least about 99.9% ee, including all values therebetween. In some embodiments, the process provides the (R)-enantiomer of a compound of Formula (I) in an enantiomeric excess of about or at least about 80% ee. In some embodiments, the process provides the (R)-enantiomer of a compound of Formula (I) in an enantiomeric excess of a least about 99.5%. In some embodiments, ee is measured by chiral HPLC.

In some embodiments, the methods of resolving a the compound of Formula (I) described herein, provides the (R)-enantiomer of a compound of Formula (I) an enantiomeric excess of from about 60% ee to about 100% ee, including from about 65% ee, about 70% ee, about 80% ee, about 85% ee, about 90% ee, about 91%, about 92%, about 93% ee, about 94% ee, about 95% ee, about 96% ee, about 97% ee, about 98% ee, about 99% ee, about 99.5% ee, about 99.9% ee to about 100% ee, including all subranges therebetween.

### NUMBERED EMBODIMENTS OF THE DISCLOSURE

In addition to the disclosure above, the Examples below, and the appended claims, the disclosure sets forth the following numbered embodiments.
1. A process for resolving the (S)- and (R)-enantiomers of a compound of Formula (I):

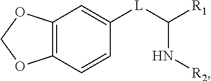

wherein L is a C₁-C₅ alkylene, carbocyclyl, or heterocyclyl;
R¹ is alkyl, benzyl or phenethyl; and
R² is hydrogen, alkyl, benzyl or phenethyl;
the process comprising:
(a) forming a diastereomeric salt comprising(S)- and (R)-enantiomers of the compound of Formula (I) by mixing the compound of Formula (I) with a chiral acid in a solvent and
(b) resolving the (S)- and (R)-enantiomers of the compound of Formula (I),
wherein the enantiomeric excess of the compound of Formula (I) is less than about 50%.
2. A process for resolving the (S)- and (R)-enantiomers of a compound of Formula (I):

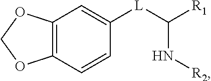

wherein L is a C₁-C₅alkylene, carbocyclyl, or heterocyclyl;
R¹ is alkyl, benzyl or phenethyl; and
R² is hydrogen, alkyl, benzyl or phenethyl;
the process comprising:
(a) mixing a compound of Formula (I) and a chiral acid in a solvent to provide a diastereomeric salt of Compound (I) and
(b) resolving the (S)- and (R)-enantiomers of the compound of Formula (I),
wherein the enantiomeric excess of the compound of Formula (I) is less than about 50%.
3. The process of embodiment 1 or 2, wherein the resolving step (b) comprises preferential crystallization of the (S)- or (R)-enantiomer of the compound of Formula (I).
4. The process of embodiment 3, comprising the preferential crystallization of the (S)-enantiomer of the compound of Formula (I).
5. The process of embodiment 3, comprising the preferential crystallization of the (R)-enantiomer of the compound of Formula (I).
6. The process of any of embodiments 1-5, wherein R¹ and R² are methyl.
7. The process of any of embodiments 1-5, wherein R¹ is methyl and R² is hydrogen.
8. The process of any one of embodiments 1-7, wherein L is methylene.
9. The process of any one of embodiments 1-6 or 8, wherein R¹ and R² are methyl and L is methylene.
10. The process of any of embodiments 1-5 or 7-8, wherein R¹ is methyl, R² is hydrogen and L is methylene.
11. The process of any of embodiments 1-10, wherein the solvent is toluene, acetonitrile, methanol, ethanol, isopropanol, dichloromethane, or mixtures thereof.
12. The process of any of embodiments 1-11, wherein the solvent is acetonitrile.
13. The process of any of embodiments 1-12, wherein the mixture of step (a) further comprises an anti-solvent.
14. The process of embodiment 13, wherein the anti-solvent is a heptane or methyl t-butyl ether.
15. The process of any of embodiments 1-14, wherein the step (a) mixture comprises about 0.5 molar equivalents of the chiral acid relative to amount of racemate.
16. The process of any of embodiments 1-15, wherein the step (a) mixture comprises about 1.0 molar equivalent of the chiral acid relative to amount of racemate.
17. The process of any of embodiments 1-16, wherein the chiral acid (S)-2-methoxy-2-phenylacetic acid, L-tartaric acid, dibenzoyl-L-tartaric acid, di-p-toluoyl-L-tartaric acid, L-ascorbic acid, L-aspartic acid, (S)-(+)-mandelic acid, (−)-di-O-isopropyledene-2-keto-L-gulonic acid or (1R, 3S)-(+)-camphoric acid.
18. The process of any of embodiments 1-16, wherein the chiral acid is (R)-2-methoxy-2-phenylacetic acid, D-tartaric acid, dibenzoyl-D-tartaric acid, di-p-toluoyl-D-tartaric acid, D-ascorbic acid, D-aspartic acid, (R)-(−)-mandelic acid, (+)-di-O-isopropyledene-2-keto-D-gulonic acid or (1S, 3R)-(−)-camphoric acid.
19. The process of any of embodiments 1-16, wherein the chiral acid is (S)-2-methoxy-2-phenylacetic acid.
20. The process of any of claims 1-16, wherein the chiral acid is (R)-2-methoxy-2-phenylacetic acid.
21. The process of any of embodiments 1-20, wherein the compound of Formula (I) is a racemate.
22. The process of any of embodiments 3-21, further comprising the step of isolating the preferentially crystallized diastereomeric salt of the compound of Formula (I).
23. The process of embodiment 22, wherein the step of isolating the diastereomeric salt is filtration.
24. The process of embodiment 22 or 23, further comprising combining the crystallized diastereomeric salt and a base to provide enantioenriched free base of the (S)- or (R)-enantiomer of the compound of Formula (I).

### EXAMPLES

Compounds of the present disclosure can be synthesized using the following exemplary methods or other methods that are known to those skilled in the art.

General reaction conditions are provided, and reaction products can be purified by known methods including silica gel chromatography using various organic solvents such as hexane, dichloromethane, ethyl acetate, methanol and the like or preparative reverse phase high pressure liquid chromatography.

Preparation of compounds can involve the protection and deprotection of various chemical groups. The need for protection and deprotection, and the selection of appropriate protecting groups can be readily determined by one skilled in the art. The chemistry of protecting groups can be found, for example, in Greene and Wuts, Protective Groups in Organic Synthesis, 44th. Ed., Wiley & Sons, 2006, as well as in Jerry March, Advanced Organic Chemistry, 4^{th} edition, John Wiley & Sons, publisher, New York, 1992 which are incorporated herein by reference in their entirety.

### Example 1: Preparation of (R)-3,4-methylenedioxymethamphetamine

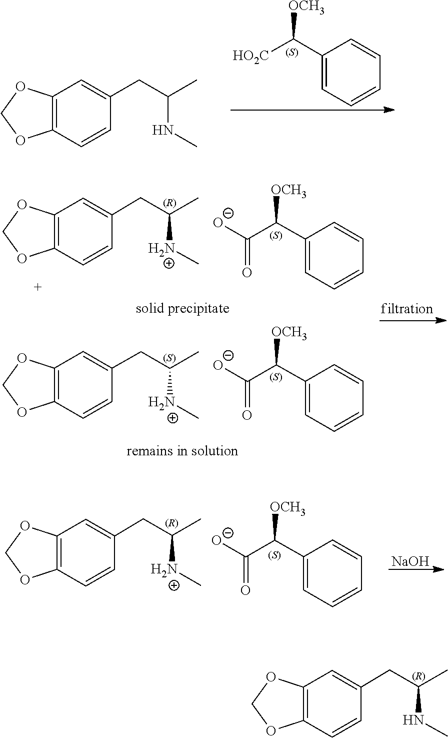

To a stirred solution of rac-MDMA (0.2 g) in acetonitrile (10 mL) at room temperature (22 to 25 °C) was added (S)-alpha-methoxy phenylacetic acid (0.5 eq) and then aged under agitation overnight. The solid precipitate was isolated by filtration to obtain 60 mg of (R)-MDMA (64% yield, and 91.5/8.5 (R/S) enantiomeric ratio as determined by Chiral HPLC analysis). When toluene was used as the solvent, the (R)-MDMA was obtained in 75% yield (of the available) and in 83.8/16.2 enantioselectivity.

1. A compound of Formula (I): 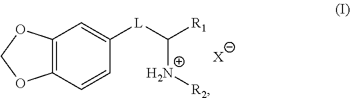
wherein:
-L- is a C₁-C₅ alkylene, carbocyclyl, or heterocyclyl;
R₁ is -alkyl, -benzyl or -phenethyl;
R₂ is -hydrogen, -alkyl, -benzyl or -phenethyl; and
X is the conjugate base of a chiral acid.

2. The compound of claim 1, wherein the chiral acid is (S)-2-methoxy-2-phenylacetic acid, L-tartaric acid, dibenzoyl-L-tartaric acid, di-p-toluoyl-L-tartaric acid, L-ascorbic acid, L-aspartic acid, (S)-(+)-mandelic acid, (−)-di-O-isopropylidene-2-keto-L-gulonic acid, (1R, 3S)-(+)-camphoric acid, (R)-2-methoxy-2-phenylacetic acid, D-tartaric acid, dibenzoyl-D-tartaric acid, di-p-toluoyl-D-tartaric acid, D-ascorbic acid, D-aspartic acid, (R)-(−)-mandelic acid, (+)-di-O-isopropylidene-2-keto-D-gulonic acid, or (1S, 3R)-(−)-camphoric acid.

3. The compound of claim 1, wherein R₁ and R₂ are -methyl.

4. The compound of claim 1, wherein R₁ is -methyl and R₂ is -hydrogen.

5. The compound of claim 1, wherein -L- is methylene.

6. The compound of claim 1, wherein R₁ and R₂ are -methyl and -L- is methylene.

7. The compound of claim 1, wherein R₁ is methyl, R₂ is -hydrogen and -L- is methylene.

8. The compound of claim 1, wherein the chiral acid is (S)-2-methoxy-2-phenylacetic acid.

9. The compound of claim 1, wherein the chiral acid is (R)-2-methoxy-2-phenylacetic acid.

10. The compound of claim 1, wherein the chiral acid is L-tartaric acid.

11. The compound of claim 1, wherein the chiral acid is D-tartaric acid.

12. The compound of claim 1, wherein the chiral acid is dibenzoyl-L-tartaric acid.

13. The compound of claim 1, wherein the chiral acid is dibenzoyl-D-tartaric acid.

14. The compound of claim 1, wherein the chiral acid is di-p-toluoyl-L-tartaric acid.

15. The compound of claim 1, wherein the chiral acid is di-p-toluoyl-D-tartaric acid.

16. The compound of claim 1, wherein the chiral acid is (S)-(+)-mandelic acid.

17. The compound of claim 1, wherein the chiral acid is (R)-(−)-mandelic acid.

18. The compound of claim 1, having an enantiomeric excess of at least about 80% ee.

19. The compound of claim 1, wherein the compound is compound of Formula (I-a): 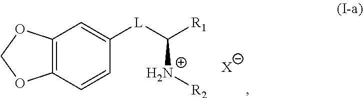
having an enantiomeric excess of at least about 80% ee.

20. The compound of claim 1, wherein the compound is compound of Formula (I-b): 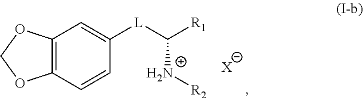
having an enantiomeric excess of at least about 80% ee.
